(19) United States
(12) Patent Application Publication
(10) Pub. No.: US 2012/0207682 A1
(43) Pub. Date: Aug. 16, 2012

(54) METHODS OF TREATING MACULAR EDEMA USING ANTIEDEMA THERAPEUTICS

(75) Inventor: Paul Ashton, Newton, MA (US)

(73) Assignee: pSivida US, Inc., Watertown, MA (US)

(21) Appl. No.: 13/372,302

(22) Filed: Feb. 13, 2012

Related U.S. Application Data
(60) Provisional application No. 61/442,151, filed on Feb. 11, 2011.

(51) Int. Cl. A61K 31/58; A61B 3/032; A61P 27/02; A61K 31/713; A61K 9/14; A61K 49/00; A61B 3/028; A61K 39/395

(52) U.S. Cl. 424/9.2; 351/246; 351/239; 424/130.1; 514/44 A; 514/174; 424/497; 424/425

(57) ABSTRACT

The invention provides methods of treating macular edema in a patient that include determining whether the patient has been diagnosed with or has experienced symptoms of macular edema for a predetermined period of time. If the patient has been diagnosed with or has experienced symptoms of macular edema for a predetermined period of time, the method of treatment includes administering a therapeutically effective amount of an AED to the patient. If the patient has not been diagnosed with or has experienced symptoms of macular edema for a predetermined period of time, the method of treatment optionally includes treating the patient with a therapy other than an AED. The invention further provides methods of treating macular edema in a patient using methods of and devices for administering an AED.

### CROSS-REFERENCE TO RELATED APPLICATIONS

This application claims the benefit of priority from U.S. Provisional Application No. 61/442,151, filed Feb. 11, 2011, the specification of which is incorporated by reference herein in its entirety.

### BACKGROUND OF THE INVENTION

The present invention relates to a novel method of treating macular edema, and more particularly to treating diabetic macular edema with an antiedema drug (AED).

Macular edema is a disease characterized by a swelling of the macula resulting from protein and fluid deposits accumulating on or under the macula, and results in blurred vision and loss of vision. Diabetic macular edema (DME), in particular, is a complication of diabetic retinopathy, which is the leading cause of blindness in working-age Americans. Approximately 4.1 million U.S. adults 40 years and older have diabetic retinopathy, about half of whom will develop DME.

Macular edema is treated with laser therapy, during which a laser is focused on the eye and multiple burns are placed in the areas of retinal leakage. However, one treatment may not be sufficient to stop the leakage of fluid around the macula, requiring patients to undergo multiple rounds of therapy. Thus, there remains a need in the art for improving methods of treating macular edema.

### SUMMARY OF THE INVENTION

The invention relates, in part, to the recognition that certain macular edema patients respond more favorably to AED therapy than others. In particular, longer-term disease and more serious impairment of visual acuity appear to correlate to more successful treatment with AED therapy, relative to alternative therapies, such as laser therapy.

Accordingly, in certain aspects, the invention provides a method of treating a patient for macular edema including determining whether the patient has been diagnosed with or has experienced symptoms of macular edema, such as DME, for a predetermined period of time. If the patient has, the method of treatment may include administering a therapeutically effective amount of an antiedema drug (AED) to the patient. If the patient has not, the method of treatment may include treating the patient with a therapy other than an AED, e.g., laser therapy.

In related embodiments where visual impairment is considered, a method of treating macular edema in a patient comprises determining whether the patient has been diagnosed with or has experienced symptoms of macular edema for a predetermined period of time and determining whether the patient has significant impairment of visual acuity, and, if the patient has experienced symptoms of macular edema for the predetermined period of time or has significant impairment of visual acuity, administering a therapeutically effective amount of an AED to the patient.

Alternatively, a method of treating macular edema in a patient comprises determining whether the patient has been diagnosed with or has experienced symptoms of macular edema for a predetermined period of time and determining whether the patient has significant impairment of visual acuity, and, if the patient has not experienced symptoms of macular edema for the predetermined period of time and does not have significant impairment of visual acuity, treating the patient with a therapy other than an AED.

In some embodiments of the invention as disclosed herein, the AED may be administered via a drug delivery device comprising a core including an AED and a polymeric skin at least partially surrounding the core. The core may comprise a matrix that includes one or more AED and a second polymer. In some such embodiments, at least one of the polymers is bioerodible. In certain embodiments, the polymeric skin is impermeable, semi-permeable, or permeable to the AED. In some embodiments, the polymeric skin itself includes an AED.

In certain embodiments of the invention as disclosed herein, the AED may be administered via a drug delivery system comprising an AED in a polymer matrix. In some embodiments, the drug delivery system may release the AED over a period of time (e.g., one week, one month, or one year). In some embodiments, the polymer matrix may be bioreodible. In some embodiments, the drug delivery system may further comprise a coating or shell that partially or wholly covers the AED and the polymer matrix.

In certain embodiments of the invention as disclosed herein, the AED may be administered via an injectable implant, microparticles, or high viscosity drug composition. In some embodiments, the implant, microparticles, or high viscosity drug composition comprises a solid polymer matrix, e.g., in which the drug is dispersed, dissolved, or suspended. In some embodiments, the matrix may be biodegradable. In some embodiments, the implant, microparticles, or high viscosity drug composition may be implanted intravitreally.

In certain embodiments of the invention as disclosed herein, the AED may be administered via a sustained release drug delivery system comprising an inner drug core that includes the AED and an inner tube covering at least a portion of the drug core. In some embodiments, the inner tube has an AED-impermeable member covering a first end of the inner tube and an AED-permeable member covering a second end of the inner tube. In these embodiments, the impermeable member may be an impermeable layer. In other embodiments, the inner tube has AED-permeable members at a first end and a second end of the inner tube. In some embodiments, the permeable member or members may be a permeable layer.

In certain embodiments of the invention as disclosed herein, the AED may be administered via a sustained-release drug delivery system comprising a silicon-containing material with a plurality of pores. In these embodiments, the AED is disposed within the pores. In some embodiments, the silicon material contains a silicon dioxide material. In some embodiments, the silicon material includes a particulate size of between about 0.1 μm and 100 μm. In some embodiments, the system may include a polymeric material capping the pores.

In embodiments employing a drug delivery device or system, the device or system can be delivered by injection or surgical implantation. In some embodiments, the device or system can be implanted at a desired location (e.g., the vitreous of the eye, under the retina, or on the sclera). In alternative embodiments, the AED is administered topically, by injection, or orally.

In other aspects, the invention relates to methods of assessing the efficacy of treatments for macular edema, especially pharmacological treatments. Thus, in one embodiment, the invention provides methods for comparing the efficacy of a therapeutic treatment in different subsets of macular edema patients, by
a) assessing the visual acuity of each of a first plurality of patients and a second plurality of patients, wherein the patients in the first plurality have been diagnosed with macular edema for at least a predetermined period of time and the patients in the second plurality have been diagnosed with macular edema for less than the predetermined period of time;
b) administering a therapeutic treatment to the first and second pluralities of patients;
c) assessing the visual acuity of each of the patients at a predetermined time point after administration;
d) comparing the visual acuities of each patient as measured before and after the therapeutic treatment to assess improvement in visual acuity during the therapeutic treatment; and
e) comparing an average improvement in visual acuity across patients in the first plurality with an average improvement in visual acuity among patients in the second plurality, thereby comparing the efficacy of the therapeutic treatment in patients diagnosed with macular edema for at least a predetermined period of time to the efficacy of the therapeutic treatment in patients diagnosed with macular edema for less than the predetermined period of time.

In related embodiments wherein visual impairment is considered, a method for comparing the efficacy of a therapeutic treatment in different subsets of macular edema patients comprises
a) assessing the visual acuity of each of a first plurality of patients and a second plurality of patients, wherein the patients in the first plurality have been diagnosed with macular edema for at least a predetermined period of time and the patients in the second plurality have been diagnosed with macular edema for less than the predetermined period of time, and subdividing each plurality two subgroups, wherein one subgroup consists of patients diagnosed as having significant impairment of visual acuity and the second subgroup consists of patients diagnosed as not having significant impairment of visual acuity;
b) administering a therapeutic treatment to the first and second pluralities of patients;
c) assessing the visual acuity of each of the patients at a predetermined time point after administration;
d) comparing the visual acuities of each patient as measured before and after the therapeutic treatment to assess improvement in visual acuity during the therapeutic treatment;
e) comparing an average improvement in visual acuity across patients in the first plurality with an average improvement in visual acuity among patients in the second plurality, thereby comparing the efficacy of the therapeutic treatment in patients diagnosed with macular edema for at least a predetermined period of time to the efficacy of the therapeutic treatment in patients diagnosed with macular edema for less than the predetermined period of time; and
f) comparing an average improvement in visual acuity across patients in the two subgroups within each plurality (or at least within the second plurality), thereby comparing the efficacy of the therapeutic treatment in patients initially suffering from significant impairment of visual acuity to the efficacy of the therapeutic treatment in patients not initially suffering from significant impairment of visual acuity.

In certain such embodiments, the predetermined time point is at least six months, e.g., approximately one year, two years, three years, three and a half years, or four years after administering the therapeutic treatment.

In other embodiments, the invention provides a method for comparing the efficacy of a therapeutic treatment in different subsets of macular edema patients, by
a) providing data representative of improvement in visual acuity before and after administration of a therapeutic treatment for a plurality of macular edema patients;
b) dividing the data into a first subset representative of patients diagnosed with macular edema for at least a predetermined period of time and a second subset representative of patients diagnosed with macular edema for less than the predetermined period of time; and
c) comparing an average improvement in visual acuity across patients in the first subset with an average improvement in visual acuity among patients in the second subset, thereby comparing the efficacy of the therapeutic treatment in patients diagnosed with macular edema for at least a predetermined period of time to the efficacy of the therapeutic treatment in patients diagnosed with macular edema for less than the predetermined period of time.

In related embodiments wherein visual impairment is considered, a method for comparing the efficacy of a therapeutic treatment in different subsets of macular edema patients comprises
a) providing data representative of improvement in visual acuity before and after administration of a therapeutic treatment for a plurality of macular edema patients;
b) dividing the data into a first subset representative of patients diagnosed with macular edema for at least a predetermined period of time and a second subset representative of patients diagnosed with macular edema for less than the predetermined period of time, and subdividing each subset into two subgroups, wherein one subgroup consists of patients diagnosed as having significant impairment of visual acuity and the second subgroup consists of patients diagnosed as not having significant impairment of visual acuity;
c) comparing an average improvement in visual acuity across patients in the first subset with an average improvement in visual acuity among patients in the second subset, thereby comparing the efficacy of the therapeutic treatment in patients diagnosed with macular edema for at least a predetermined period of time to the efficacy of the therapeutic treatment in patients diagnosed with macular edema for less than the predetermined period of time; and
d) comparing an average improvement in visual acuity across patients in the two subgroups within each subset (or at least within the second subset), thereby comparing the efficacy of the therapeutic treatment in patients initially suffering from significant impairment of visual acuity to the efficacy of the therapeutic treatment in patients not initially suffering from significant impairment of visual acuity.

In yet other embodiments, the invention provides a method for assessing the efficacy of a candidate therapeutic treatment for macular edema, comprising
a) obtaining patient characteristic data for a plurality of candidate patients diagnosed with macular edema, the patient characteristic data including the length of time for which the patient has been diagnosed with macular edema;
b) selecting test patients from the candidate patients based in part on the length of time for which each patient has been diagnosed with macular edema;
c) dividing the test patients into a first plurality of control patients and a second plurality of treatment patients;
d) assessing the visual acuity of each patient;
e) administering the candidate therapeutic treatment to the treatment patients but not to the control patients;
f) assessing the visual acuity of each of the patients at a predetermined time point after administering the candidate therapeutic treatment to the treatment patients;
g) comparing the visual acuities of each patient as measured before and after administering the candidate therapeutic treatment to the treatment patients to assess improvement in visual acuity between the assessments; and
h) comparing an average improvement in visual acuity across treatment patients with an average improvement in visual acuity among control patients, thereby assessing the efficacy of the candidate therapeutic treatment.

In related embodiments wherein visual impairment is considered, a method for assessing the efficacy of a candidate therapeutic treatment for macular edema comprises
a) obtaining patient characteristic data for a plurality of candidate patients diagnosed with macular edema, the patient characteristic data including the length of time for which the patient has been diagnosed with macular edema and the level of impairment of visual acuity;
b) selecting test patients from the candidate patients based in part on the length of time for which each patient has been diagnosed with macular edema and based in part on the significance of impairment of visual acuity (preferably selecting patients that meet a predetermined criterion for length of time, significance of impairment, or both);
c) dividing the test patients into a first plurality of control patients and a second plurality of treatment patients;
d) assessing the visual acuity of each patient;
e) administering the candidate therapeutic treatment to the treatment patients but not to the control patients;
f) assessing the visual acuity of each of the patients at a predetermined time point after administering the candidate therapeutic treatment to the treatment patients;
g) comparing the visual acuities of each patient as measured before and after administering the candidate therapeutic treatment to the treatment patients to assess improvement in visual acuity between the assessments; and
h) comparing an average improvement in visual acuity across treatment patients with an average improvement in visual acuity among control patients, thereby assessing the efficacy of the candidate therapeutic treatment.

In certain such embodiments, selecting test patients comprises selecting test patients such that at least 25%, 40%, 50%, 60%, or even 75% of the test patients have experienced symptoms of macular edema for at least a predetermined period of time and/or suffer from significant impairment of visual acuity as described below.

In any of the various methods for assessing the efficacy of treatments for macular edema, instead of distinguishing patients on having been diagnosed with macular edema for a predetermined period of time, the patients can be distinguished on the basis of having experienced symptoms of macular edema for a predetermined period of time. In either scenario, the predetermined period of time may be approximately 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

In certain embodiments of the methods described herein, the macular edema may be diabetic macular edema.

Assessing visual acuity may be performed by any suitable method, , using an eye chart, such as a Snellen chart, an early treatment of diabetic retinopathy study (ETDRS) chart, or a multiple letter acuity chart (MLAC). The need for standardized visual acuity measurements has been explained and discussed by Camparini et al. in Investigative Ophthalmology & Visual Science, May 2001, Vol. 42, No. 6, page 1226 and references cited therein. The early treatment of diabetic retinopathy study (ETDRS) chart includes the same number of letters per row (five letters per row), with equal spacing of the rows on a logarithmic scale (the rows are separated by 0.1 log unit). The row for normal vision i.e., 20/20 vision corresponds to 0 on the logarithmic scale and is the fourth row from the bottom of the chart. Hence, if the chart is expanded to 20/800 vision (16 lines above 20/20 vision) to comprise 100 letters, a patient with normal vision would be able to read at least 85 letters (16 times 5 letters in addition to the 5 letters in the 20/20 row). On this basis, the impairment in visual acuity or the improvement in visual acuity, expressed in a number of letters, can also be expressed as a percentage relative to normal vision.

Accordingly, a person able to read 20 letters has a visual acuity corresponding to 23.5% of normal; a person able to read 25 letters has a visual acuity corresponding to 29.4% of normal; a person able to read 30 letters has a visual acuity corresponding to 35.3% of normal; a person able to read 40 letters has a visual acuity corresponding to 47.0% of normal; a person able to read 43 letters has a visual acuity corresponding to 50.5% of normal; a person able to read 45 letters has a visual acuity corresponding to 52.9% of normal; and a person able to read 49 letters has a visual acuity corresponding to 57.6% of normal. In various embodiments, any or all such people may be considered to have significant impairment of visual acuity.

In certain embodiments, a person able to read 50 letters has a visual acuity corresponding to 58.5% of normal; a person able to read 55 letters has a visual acuity corresponding to 64.7% of normal; a person able to read 60 letters has a visual acuity corresponding to 70.6% of normal; a person able to read 63 letters has a visual acuity corresponding to74.1% of normal; a person able to read 65 letters has a visual acuity corresponding to 76.5% of normal; and a person able to read 68 letters has a visual acuity corresponding to 80.0% of normal. In various embodiments, any or all such people may be considered not to have significant impairment of visual acuity.

In various embodiments, patients are deemed to have significant impairment of visual acuity if their visual acuity is below 45% of normal, below 50% of normal, below 55% of normal, below 60% of normal, or below 65% of normal.

In various embodiments, patients are deemed to have significant impairment of visual acuity if, on an early treatment of diabetic retinopathy study (ETDRS) chart, they can read no more than 0 letters, no more than 1 letter, no more than 2 letters, no more than 3 letters, no more than 4 letters, no more than 5 letters, no more than 6 letters, no more than 7 letters, no more than 8 letters, no more than 9 letters, no more than 10 letters, no more than 11 letters, no more than 12 letters, no more than 13 letters, no more than 14 letters, no more than 15 letters, no more than 16 letters, no more than 17 letters, no more than 18 letters, no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters, no more than 49 letters, no more than 50 letters, no more than 51 letters, no more than 52 letters, no more than 53 letters, no more than 54 letters, or no more than 55 letters.

In certain preferred embodiments, patients are deemed to have significant impairment of visual acuity if, on an early treatment of diabetic retinopathy study (ETDRS) chart, they can read no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters, no more than 49 letters, no more than 50 letters, no more than 51 letters, or no more than 52 letters.

In certain preferred embodiments, where both visual impairment and length of time of diagnosis or symptoms are considered, a patient is deemed to have significant impairment of visual acuity if his visual acuity is below 55% of normal, most preferably below 58% of normal, while the predetermined amount of time is approximately 36 months (3 years).

### DETAILED DESCRIPTION OF THE INVENTION

Macular edema is thought to be caused by an up-regulation of certain growth factors including vascular endothelial growth factor. There are several anti-edematous drugs (AEDs) being developed to treat macular edema. Some drugs inhibit VEGF, for example with antibodies and antibody fragments that bind to VEGF. Other drugs in development (such as corticosteroids) have multiple targets, including VEGF.

The present invention stems from the recognition that certain subsets of DME patients respond better to AED therapies than others, e.g., depending on the length of time the DME and/or its symptoms have persisted. Accordingly, the present invention provides methods of treating macular edema in a patient comprising determining whether the patient has experienced symptoms of macular edema for a predetermined period of time or has been diagnosed with macular edema for a predetermined period of time. In some embodiments, if the patient has experienced symptoms of macular edema for the predetermined period of time or has been diagnosed with macular edema for the predetermined period of time, the patient is administered a therapeutically effective amount of an AED, such as a corticosteroid (e.g., a glucocorticoid). Alternatively, if the patient has not experienced symptoms of macular edema for the predetermined period of time or has not been diagnosed with macular edema for the predetermined period of time, the patient may be treated with a therapy other than an AED, such as laser therapy. The predetermined period of time may be approximately 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

Symptoms of macular edema can include, but are not limited to, distorted vision, blurred vision, loss of vision, protein deposits on or under the macula, the accumulation of fluid under the macula, and thickening or swelling of the macula. In certain embodiments, the macular edema is diabetic macular edema (DME).

In various embodiments, patients are deemed to have significant impairment of visual acuity if, on an early treatment of diabetic retinopathy study (ETDRS) chart, they can read no more than 0 letters, no more than 1 letter, no more than 2 letters, no more than 3 letters, no more than 4 letters, no more than 5 letters, no more than 6 letters, no more than 7 letters, no more than 8 letters, no more than 9 letters, no more than 10 letters, no more than 11 letters, no more than 12 letters, no more than 13 letters, no more than 14 letters, no more than 15 letters, no more than 16 letters, no more than 17 letters, no more than 18 letters, no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

In certain embodiments, patients are deemed to have significant impairment of visual acuity if, on an early treatment of diabetic retinopathy study (ETDRS) chart, they can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

The term “patient,” as used herein, refers to either a human or a non-human animal, preferably a mammal.

An AED may include any therapeutic that treats edema. Examples of AEDs include antibodies (, bevacizumab (Avastin™), ranibizumab (Lucentis™), and infliximab (Remicade™)), anti-inflammatory treatments (e.g., NSAIDs), cortisone, indomethacin, nepafenac, anti-VEGF agents, pegaptanib sodium (Macugen™), choline fenofibrate, bevasiranib, rapamycin, minocycline, mecamylamine, keterolac tromethamine, siRNA compounds, and denufosol tetrasodium. A therapeutically effective amount of an AED will depend on the AED being administered. Therapeutically effective amounts of various AEDs are well known to one of skill in the art, and/or can be ascertained by any suitable means.

Corticosteroids are a family of compounds that include the adrenal steroid hormone cortisol (hydrocortisone) and related synthetic drugs, including, but not limited to, betamethasone, beclomethasone, beclomethasone dipropionate, budesonide, clobetasol, cortisol, cortisone, dexamethasone, fludrocortisone, flunisolide, flunisolide hemihydrate, fluocinolone, fluocinolone acetonide, fluocinonide, fluticasone, fluticasone propionate, methylprednisolone, mometasone, mometasone furoate anhydrous, mometasone furoate monohydrate, prednisone, prednisolone, triamcinolone, and triamcinolone acetonide, any of which can be used in the methods and uses described herein. Corticosteroids generally have similar mechanisms of action: they bind to specific corticosteroid binding proteins in the cytoplasm. These complexes are then transported into the nucleus where they bind to discrete portions of the cell's DNA. In certain embodiments of the methods and uses described herein, the corticosteroid is selected from: betamethasone, budesonide, cortisol, cortisone, dexamethasone, fludrocortisone, fluocinolone, fluocinolone acetonide, prednisolone, prednisone, methylprednisolone, and triamcinolone. A therapeutically effective amount of a corticosteroid will depend on the corticosteroid being administered. Therapeutically effective amounts of corticosteroids are well known to one of skill in the art, and can be assayed for by any means known in the art.

In certain embodiments, the AED is administered topically at a desired location, e.g., the sclera or the cornea. In other embodiments, the AED is administered by injection, e.g., intravitreal, periocular, intraocular, or intravenous injection. In still other embodiments, the AED can be administered via biophoresis.

In certain embodiments, the AED is administered via a drug delivery device that releases the AED locally over a period of time. In certain embodiments, the device is inserted into the patient at a desired location. This location can be the vitreous of the eye, under the retina, or on the sclera. In certain embodiments, the device is shaped and sized for injection, e.g., intraocular injection. In some embodiments, the device is inserted into the patient's eye by injection or surgical implantation.

In some embodiments, the device includes a core containing one or more polymers, as described in International Patent Application No. PCT/US04/35430, incorporated herein by reference for the devices disclosed therein. The core may be surrounded by one or more polymer outer layers (referred to herein as “coatings,” “skins,” or “outer layers”). In some embodiments, the core may be coated with one or more polymer coatings. The device is preferably configured to provide for controlled release of the AED(s) for an extended period, e.g., at least a week, at least a month, or at least six months.

In embodiments wherein the core is surrounded by a skin, the skin may be permeable, semi-permeable, or impermeable to the drug, or to the fluid environment to which the device may be exposed. The drug core may include a polymer matrix which does not significantly affect the release rate of the drug. Alternatively, the polymer matrix may affect the release rate of the drug. The skin, the polymer matrix of the drug core, or both may be bioerodible. The device may be fabricated as an extended mass that is segmented into drug delivery devices, which may be left uncoated so that the drug core is exposed on all sides or (where a skin is used) at the ends of each segment, or coated with a layer such as a layer that is permeable to the drug, semi-permeable to the drug, impermeable, or bioerodible.

Suitable materials to form the skin and the core are numerous. In this regard, U.S. Pat. No. 6,375,972, by Hong Guo et al., incorporated by reference herein for the devices and materials disclosed therein, describes a number of suitable materials for forming implantable drug delivery devices, which materials may be more specifically used for injectable drug delivery devices. Biocompatible materials may be selected for the materials which will, when the drug delivery device is fully constructed, come in contact with the patient's biological tissues. Suitable polymers for use include, but are not limited to, poly(caprolactone) (PCL), ethylene vinyl acetate polymer (EVA), poly(vinyl alcohol) (PVA), poly(ethylene glycol) (PEG), poly(vinyl acetate) (PVAC), poly(lactic acid) (PLA), poly(glycolic acid) (PGA), poly(lactic-co-glycolic acid) (PLGA), polyalkyl cyanoacrylate, polyurethane, nylons, or copolymers thereof In polymers including lactic acid monomers, the lactic acid may be D-, L-, or any mixture of D- and L- is

Material(s) used to form the inner drug core may be selected so that the activity of the AED in the core of the product is sufficient for producing the desired effect when injected. Furthermore, when the AED is admixed with a polymer for forming a matrix in the core, the polymer material which forms the matrix may be advantageously selected so that the AED is not destabilized by the matrix. The matrix material may be selected so that diffusion through the matrix has little or no effect on the release rate of the AED from the matrix. Also, the particle size of the AED(s) used in the matrix may be selected to modulate the rate of dissolution of the AED(s), in turn affecting the release rate of the AED from the device.

The materials of the drug delivery device may be selected to be stable during the release period for the drug delivery device. The materials may optionally be selected so that, after the drug delivery device has released the drug for a predetermined amount of time, the drug delivery device erodes in situ, i.e., it is bioerodible. The materials may also be selected so that, for the desired life of the delivery device, the materials are stable and do not significantly erode, and the pore size of the materials does not change. Optionally, the materials may be chosen to be bioerodible at rates that control, or contribute to control of, the release rate of any active agents. It will be appreciated that other materials, such as additional coatings on some of or the entire device may be similarly selected for their bioerodible properties.

In some embodiments, the core comprises a matrix of the one or more AEDs and a second one or more polymer. In some embodiments, at least one of the second one or more polymers is bioerodible. In some embodiments, polymers employed within the skin and the core, or coatings added to the skin and/or core, is selected with respect to permeability to one or more drugs within the core. Permeability is necessarily a relative term. As used herein, the term “permeable” is intended to mean permeable or substantially permeable to a substance, which is typically the drug that the device delivers unless otherwise indicated (for example, where a membrane is permeable to a biological fluid from the environment into which a device is delivered). As used herein, the term “impermeable” is intended to mean impermeable or substantially impermeable to substance, which is typically the drug that the device delivers unless otherwise indicated (for example, where a membrane is impermeable to a biological fluid from the environment into which a device is delivered). The term “semi-permeable” is intended to mean selectively permeable to some substances but not others. It will be appreciated that in certain cases, a membrane may be permeable to a drug, and also substantially control a rate at which the drug diffuses or otherwise passes through the membrane. Consequently, a permeable membrane may also be a release-rate-limiting or release-rate-controlling membrane, and in certain circumstances, permeability of such a membrane may be one of the most significant characteristics controlling release rate for a device. Thus, if part of a device is coated by a permeable coating and the rest of the device is covered by an impermeable coating, it is contemplated that, even though some drug may pass through the impermeable coating, the drug will predominately be released through the part of the device coated only with the permeable coating. In certain embodiments, the polymeric skin of the device is impermeable, semi-permeable, or permeable to at least one of the one or more AEDs. In certain embodiments, the polymeric skin of the device comprises at least one AED. In certain embodiments, at least one polymer is bioerodible.

In certain embodiments, the device is shaped and sized for injection, e.g., through at least one of a cannula having a size from about 30 gauge to about 15 gauge or a needle having a size from about 30 gauge to about 15 gauge, more preferably, with a cannula or needle having a size from about 27 gauge to about 25 gauge. In certain embodiments, the injection can be periocular or intraocular.

In some embodiments, an AED may be administered via a sustained release drug delivery system as described in International Patent Application No. PCT/US01/12700, incorporated herein by reference. Preparing a device using a dimensionally-stable tube (e.g., a polymeric tube) to hold the drug reservoir allows for significantly easier handling of the tube and reservoir during manufacture, because the tube fully supports both its own weight and the weight of the reservoir. Thus, the tube is not a coating, because a coating cannot support its own weight. Also, this rigid structure allows the use of drug slurries drawn into the tube, which allows the fabrication of longer cylindrical devices. During use of such systems, the tube which holds the drug reservoir is sufficiently strong or rigid to maintain a substantially constant diffusion area as drug (e.g., AED) diffuses out of the device so that the diffusion rate from the device does not change substantially because of the change in the drug reservoir.

Thus, in certain embodiments wherein an AED is administered via a sustained release drug delivery system, the sustained release drug delivery system comprises an inner drug core comprising an AED and an inner tube impermeable to the passage of the AED, wherein the inner tube has first and second ends and covering at least a portion of said inner drug core. In these embodiments, the inner tube is sized and formed of a material so that the inner tube is dimensionally stable to accept the drug core without changing shape. In certain such embodiments, the system also comprises an impermeable member covering the inner tube first end that prevents passage of the AED out of the drug core through the inner tube first end and a permeable member covering the inner tube second end that allows diffusion of the AED out of the drug core through the inner tube second end. In alternative such embodiments, the system comprises permeable members covering the inner tube first end and the inner tube second end that allow diffusion of the AED out of the drug core through the inner tube first end and second end. In embodiments comprising an impermeable member, the impermeable member can comprise an impermeable cap abutting the inner tube first end. Materials suitable for the construction of these systems are described supra and below.

In some such embodiments, the inner tube comprises a polymer. In some embodiments, the sustained release drug delivery system further comprises a permeable outer layer covering the inner tube first end, second end, or both.

In some embodiments, an AED is administered via a composition as described in International Patent Application No. PCT/US08/69474, incorporated herein by reference for the disclosed particles and other delivery devices. In these embodiments, the composition comprises a silicon-containing material, such as silicon and/or silicon dioxide, comprising a plurality of pores and an AED disposed within the pores. Porous silicon and silica are biocompatible and can be eroded in, or resorbed into, a patient without significant detrimental effect.

In some embodiments, the silicon material of the composition comprises a silicon dioxide material. In some embodiments, the composition comprises a particulate size of between about 0.1 μm and 100 μm. In some embodiments, the composition further comprises a polymeric material capping the pores. Materials useful in the manufacture of the composition are described supra.

In some embodiments, an AED is administered via biodegradable drug-eluting particles for sustained release of the AED as described in International Patent Application No. PCT/US2010/033541, incorporated herein by reference for the devices and materials disclosed therein. The particles are designed to release the AED in a controlled fashion and then biodegrade in the body, thereby avoiding the need for surgical resection of the particles. In certain embodiments, the drug-eluting particles of the invention are formed from silicon that is made biodegradable through porosification. In certain embodiments, the particle comprises a porous silicon body comprising a reservoir formed within the porous silicon body having at least one opening to an exterior of the body, wherein the reservoir contains a drug core comprising the AED, and an AED-permeable seal disposed over the at least one opening. In certain embodiments, the seal provides for sustained release of the AED when the particle is administered to the patient. Once the particle is administered to the patient, the AED diffuses through the permeable seal of the particle in a sustained fashion. The porous silicon body and agent-permeable seal that remain in the body after the useful life (e.g., therapeutic utility) of the particle has ended, erode under normal biological conditions to biocompatible byproducts. Representative devices comprising porous silicon may be seen in U.S. Pat. No. 6,770,480, which is incorporated herein by reference for the devices and materials disclosed therein.

This invention will be better understood from the Example which follows. However, one skilled in the art will readily appreciate that the specific methods and results discussed are merely illustrative of the invention as described more fully in the embodiments which follow thereafter.

### EXAMPLES

### Example 1

An intraocular implant that releases fluocinolone acetonide (“FA”) was prepared in two variant formulations. Both were prepared by loading a paste containing 180 μg of FA in a 10% polyvinyl alcohol (“PVA”) solution (i.e., 90% FA particles, 10% PVA-containing liquid) into the center of a 3.5 mm-long polyimide tube having an inner diameter of 0.0135 inches. The polyimide tube was open at both ends. In a high-release rate formulation, each end was coated with a 10% PVA solution to form a PVA end cap. In a low-release rate formulation, only one end is coated with the 10% PVA solution to form a PVA end cap and the other end is sealed with an impermeable silicone adhesive.

956 patients with clinically significant diabetic macular edema were recruited into two phase three clinical trials. For inclusion in the clinical trials, patients were r29528597_{—}2equired to meet the following criteria: having at least one round of laser therapy not sooner than 6 months before being entered into the study and having a best corrected visual acuity of between 19 and 68 letters, inclusive, on an early treatment of diabetic retinopathy study chart (ETDRS). Patients were randomized to receive either a low dose intraocular implant (releasing fluocinolone acetone), a high dose intraocular implant, or a sham procedure. After 6 weeks patients were eligible to receive additional laser treatments as indicated. In the trial, 184 patients were randomized to control, and 375 to low dose.

After two years, 28.7% of the patients receiving the low dose intraocular implant gained 15 or more letters on an ETDRS, compared with 16.2% of patients randomized to the control. By three years, the comparison was 28.7% versus 18.9% (a difference of approximately 10% at each time point). With respect to mean visual acuity, control patients gained approximately 2.0 letters after three years, while patients treated with the low dose device gained 5.3 letters, a mean benefit of 3.3 letters.

Closer examination of the results showed that in the half of the patients who had been diagnosed with DME for a shorter period of time (less than approximately 3.5 years), the low dose was no more effective, and possibly less effective, than the control. At two years, 20.8% of patients randomized to the control gained 15 or more letters versus 21.7% of patients randomized to receive the low dose device. By three years, this was 27.8% of the control versus 22.3% of the patients receiving the low dose device.

By contrast, in the other half of patients (i.e., who had been diagnosed with DME for more than 3.5 years), only 13.4% of control patients and 34.4% of low dose had gained 15 or more letters. At three years, these comparisons remained essentially unchanged at 13.4% for the control and 34.0% for patients receiving the low dose (a difference of over 20%).

Similar results were observed for mean vision gained. On average, the half of patients who had had DME for a shorter duration did as well with either the sham procedure or drug treatment. At month 24, patients in the control group had a mean change of 1 letter versus 2.4 letters for patients receiving the drug, a clinically insignificant difference. At three years, these values were 2.3 letters in the control group versus 2.4 letters in the drug group. By contrast, in the half of patients who had had DME for a longer duration, the mean gain at 24 months was 2.2 letters in the control group and 8.0 letters in patients receiving the low dose device (a difference of 5.8 letters). The mean gain at 36 months was 1.8 letters in control patients and 7.6 letters in patients receiving the low dose device (also a difference of 5.8 letters).

These data demonstrate that a patient with macular edema (e.g., diabetic macular edema) can be evaluated to determine an appropriate course of therapy depending on the length of time that the patient has had the macular edema. Because patients with short-term disease are less responsive to corticosteroid therapy, the effectiveness of corticosteroid treatment on longer-term patients is partially masked when data is evaluated only in the general population of macular edema patients, without regard to the long- or short-term nature of the disease in individual patients. These findings are broadly applicable to treatment with a variety of AEDs.

### Example 2

Grouping the patients who had DME for over three years, whether they had significant impairment of visual acuity or not, together with people with DME for less than 3 years and with significant impairment of visual acuity, the following results are obtained:

Study 1: Proportion of subjects with ≧15 letter improvement in visual acuity

| Time | | | t-test |
|---|---|---|---|
| (mo) | Sham | Low Dose | (two sided p) |
| 24 | 8/66 (12.1%) | 45/132 (34%) | 0.0002 |
| 30 | 8/66 (12.1%) | 48/132 (36.3%) | <0.00001 |
| 36 | 11/66 (16.6%) | 45/132 (34%) | 0.0054 |

Study 2: Proportion of subjects with ≧15 letter improvement in visual acuity

| Time | | | | t-test |
|---|---|---|---|---|
| (mo) | Sham | Low Dose | | (two sided p) |
| 24 | 12/62 (19.3%) | 45/121 | (37.2%) | 0.008 |
| 30 | 9/62 (14.5%) | 50/121 | (41.3%) | <0.00001 |
| 36 | 10/62 (16.1%) | 44/121 | (36..4% | 0.0018 |

Studies 1 and 2 combined: Proportion of subjects with ≧15 letter improvement in visual acuity

| Time | | | t-test |
|---|---|---|---|
| (mo) | Sham | Low Dose | (two sided p) |
| 24 | 14/128 (10.9%) | 90/253 (35.6%) | <0.00001 |
| 30 | 17/128 (13.2%) | 98/253 (38.7%) | <0.00001 |
| 36 | 21/128 (16.4%) | 89/253 (35.2%) | <0.00001 |

What is claimed is:

1. A method of treating macular edema in a patient, comprising
determining whether the patient has experienced symptoms of macular edema for a predetermined period of time; or
determining whether the patient has significant impairment of visual acuity; or
determining both; and
if the patient has experienced symptoms of macular edema for the predetermined period of time or, optionally, has significant impairment of visual acuity, administering a therapeutically effective amount of an AED to the patient.

2. A method of treating macular edema in a patient, comprising
determining whether the patient has experienced symptoms of macular edema for a predetermined period of time;
optionally determining whether the patient has significant impairment of visual acuity; and
if the patient has not experienced symptoms of macular edema for the predetermined period of time and, optionally, does not have significant impairment of visual acuity, treating the patient with a therapy other than an AED.

3. A method of treating macular edema in a patient, comprising
determining whether the patient has been diagnosed with macular edema for a predetermined period of time; or
determining whether the patient has significant impairment of visual acuity; or
determining both; and
if the patient has been diagnosed with macular edema for the predetermined period of time, or, optionally, has significant impairment of visual acuity, administering a therapeutically effective amount of an AED to the patient.

4. A method of treating macular edema in a patient, comprising
determining whether the patient has been diagnosed with macular edema for a predetermined period of time; and
optionally determining whether the patient has significant impairment of visual acuity; and,
if the patient has not been diagnosed with macular edema for the predetermined period of time and, optionally, does not have significant impairment of visual acuity, treating the patient with a therapy other than an AED.

5. The method of any claim 2 or 4, wherein the therapy comprises laser therapy.

6. The method of any preceding claim, wherein the macular edema is diabetic macular edema.

7. The method of any preceding claim, wherein the AED is selected from: antibodies, anti-inflammatory treatments, NSAIDs, anti-VEGF agents, siRNA compounds, corticosteroids, and glucocorticoids.

8. The method of any of claims 1-6, wherein the AED is selected from: bevacizumab, ranibizumab, infliximab, cortisone, indomethacin, nepafenac, pegaptanib sodium, choline fenofibrate, bevasiranib, rapamycin, minocycline, mecamylamine, keterolac tromethamine, denufosol tetrasodium betamethasone, beclomethasone, beclomethasone dipropionate, budesonide, clobetasol, cortisol, cortisone, dexamethasone, fludrocortisone, flunisolide, flunisolide hemihydrate, fluocinolone, fluocinolone acetonide, fluocinonide, fluticasone, fluticasone propionate, methylprednisolone, mometasone, mometasone furoate anhydrous, mometasone furoate monohydrate, prednisone, prednisolone, triamcinolone, and triamcinolone acetonide.

9. The method of any preceding claim, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

10. The method of any preceding claim, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters, no more than 49 letters, no more than 50 letters, no more than 51 letters, or no more than 52 letters.

11. The method of any preceding claim, wherein significant impairment is characterized by the fact that the patient has a visual acuity corresponding to below 23.5% of normal, below 29.4% of normal, below 35.3% of normal, below 47.0% of normal, 50% of normal, below 50.5% of normal, below 52.9% of normal, below 55% of normal, below 57.6% of normal, or below 58% of normal.

12. The method of any preceding claim, wherein the AED is administered via a sustained-release composition comprising one or more AEDs disposed in a polymer matrix.

13. The method of claim 10, wherein the composition further comprises a polymeric layer at least partially surrounding the core.

14. The method of claim 11, wherein at least one of the polymeric layer and the polymer matrix is bioerodible.

15. The method of any of claims 12-14, wherein the polymer matrix comprises at least one of poly(vinyl acetate) (PVAC), poly(caprolactone) (PCL), polyethylene glycol (PEG), poly(dl-lactide-co-glycolide) (PLGA), ethylene vinyl acetate polymer (EVA), poly(vinyl alcohol) (PVA), poly(lactic acid) (PLA), poly(glycolic acid) (PGA), polyalkyl cyanoacrylate, polyurethane, or nylon, or a copolymer thereof.

16. The method of any of claims 12-15, wherein the polymeric layer is impermeable, semi-permeable, or permeable to at least one of the one or more AEDs.

17. The method of any of claims 12-16, wherein the polymeric skin comprises at least one of PVAC, PCL, PEG, PLGA, EVA, PVA, PLA, PGA, polyalkyl cyanoacrylate, polyurethane, or nylon, or a copolymer thereof.

18. The method of any of claims 12-17, wherein at least one of the first one or more polymers and the second one or more polymers is bioerodible.

19. The method of any of claims 12-18, wherein the polymeric skin further comprises at least one corticosteroid.

20. The method of any of claims 12-19, wherein the device is shaped and sized for injection through at least one of a cannula having a size from about 30 gauge to about 15 gauge or a needle having a size from about 30 gauge to about 15 gauge.

21. The method of any of claims 12-20, wherein the device is shaped and sized for at least one of periocular or intraocular injection.

22. The method of any of claims 12-21, wherein the device provides sustained release of the one or more drugs when exposed to a biological medium.

23. The method of any of claims 12-22, wherein administration comprises inserting the device into the patient at a desired location.

24. The method of claim 23, wherein inserting comprises injecting the device at a desired location.

25. The method of claim 23, wherein inserting comprises surgically implanting the device at a desired location.

26. The method of any of claims 23-25, wherein the location is selected from the vitreous of the eye, under the retina, and on the sclera.

27. The method of any of claims 1-9, wherein the AED is administered via a sustained release drug delivery system comprising:
an inner drug core comprising an AED;
an inner tube impermeable to the passage of said corticosteroid, said inner tube having first and second ends and covering at least a portion of said inner drug core, said inner tube sized and formed of a material so that said inner tube is dimensionally stable to accept said drug core without changing shape;
an impermeable member covering said inner tube first end, said impermeable member preventing passage of said corticosteroid out of said drug core through said inner tube first end; and
a permeable member covering said inner tube second end, said permeable member allowing diffusion of said corticosteroid out of said drug core through said inner tube second end.

28. The method of any of claims 1-9, wherein said method comprises administering a sustained release drug delivery system comprising:
an inner drug core comprising an AED;
an inner tube impermeable to the passage of said corticosteroid, said inner tube having first and second ends and covering at least a portion of said inner drug core, said inner tube sized and formed of a material so that said inner tube is dimensionally stable to accept said drug core without changing shape; and
permeable members covering said inner tube first end and said inner tube second end, said permeable members allowing diffusion of said corticosteroid out of said drug core through said inner tube first end and said inner tube second end.

29. The method of claim 26 or 28, wherein the inner tube comprises a polymer.

30. The method of claim 26, wherein said impermeable member comprises an impermeable layer covering the inner tube first end.

31. The method of claim 26 or 28, wherein said permeable member comprises a permeable layer that covers the inner tube first end, second end, or both.

32. The method of claim 31, further comprising a permeable outer layer surrounding at least a portion of the inner tube.

33. The method of any of claims 27-32, wherein administration comprises the step of inserting the system into the patient at a desired location.

34. The method of claim 33, wherein inserting comprises injecting the system at a desired location.

35. The method of claim 33, wherein inserting comprises surgically implanting the system at a desired location.

36. The method of any of claims 33-35, wherein the location is selected from the vitreous of the eye, under the retina, and onto the sclera.

37. The method of any of claims 1-9, wherein the AED is administered via a composition comprising:
a silicon-containing material comprising a plurality of pores; and
an AED disposed within the pores.

38. The method of claim 37, wherein the silicon material comprises a silicon dioxide material.

39. The method of any of claim 37 or 38, wherein the composition comprises a particulate size of between about 0.1 μm and 100 μm.

40. The method of any of claims 37-39, wherein the composition further comprises a polymeric material capping the pores.

41. The method of any of claims 1-9, wherein the AED is administered topically at a desired location.

42. The method of claim 41, wherein the location is selected from the sclera and the cornea.

43. The method of any one of claims 1-9, wherein the AED is administered by injection.

44. The method of claim 43, wherein the injection is selected from intravitreal injection, periocular injection, intraocular injection, and intravenous injection.

45. The method of any of claims 1-9, wherein the AED is administered orally.

46. A method for comparing the efficacy of a therapeutic treatment in different subsets of macular edema patients, comprising
a) assessing the visual acuity of each of a first plurality of patients and a second plurality of patients, wherein the patients in the first plurality have been diagnosed with macular edema for at least a predetermined period of time and the patients in the second plurality have been diagnosed with macular edema for less than the predetermined period of time, optionally wherein each plurality is subdivided into two subgroups, wherein one subgroup consists of patients diagnosed as having significant impairment of visual acuity and the second subgroup consists of patients diagnosed as not having significant impairment of visual acuity;
b) administering a therapeutic treatment to the first and second pluralities of patients;
c) assessing the visual acuity of each of the patients at a predetermined time point after administration;
d) comparing the visual acuities of each patient as measured before and after the therapeutic treatment to assess improvement in visual acuity during the therapeutic treatment;
e) comparing an average improvement in visual acuity across patients in the first plurality with an average improvement in visual acuity among patients in the second plurality, thereby comparing the efficacy of the therapeutic treatment in patients diagnosed with macular edema for at least a predetermined period of time to the efficacy of the therapeutic treatment in patients diagnosed with macular edema for less than the predetermined period of time; and
f) optionally comparing an average improvement in visual acuity across patients in the subgroups within each plurality.

47. The method of claim 46, wherein the predetermined time point is at least one year after administering the therapeutic treatment.

48. The method of claim 46 or 47, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

49. The method of any claims 46-48, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

50. The method of any claims 46-49, wherein significant impairment is characterized by the fact that the patient has a visual acuity below 23.5% of normal, below 29.4% of normal, below 35.3% of normal, below 47.0% of normal, below 50% of normal, below 50.5% of normal, below 52.9% of normal, below 55% of normal, below 57.6% of normal, or below 58% of normal.

51. The method of any of claims 46-50, wherein assessing visual acuity comprises using an eye chart, such as a Snellen chart, an early treatment of diabetic retinopathy study (ETDRS) chart, or a multiple letter acuity chart (MLAC).

52. The method of any of claims 46-51, wherein the macular edema is diabetic macular edema.

53. A method for comparing the efficacy of a therapeutic treatment in different subsets of macular edema patients, comprising
a) providing data representative of improvement in visual acuity before and after administration of a therapeutic treatment for a plurality of macular edema patients;
b) dividing the data into a first subset representative of patients diagnosed with macular edema for at least a predetermined period of time and a second subset representative of patients diagnosed with macular edema for less than the predetermined period of time, optionally wherein each subset is subdivided into two subgroups, wherein one subgroup consists of patients diagnosed as having significant impairment of visual acuity and the second subgroup consists of patients diagnosed as not having significant impairment of visual acuity;
c) comparing an average improvement in visual acuity across patients in the first subset with an average improvement in visual acuity among patients in the second subset, thereby comparing the efficacy of the therapeutic treatment in patients diagnosed with macular edema for at least a predetermined period of time to the efficacy of the therapeutic treatment in patients diagnosed with macular edema for less than the predetermined period of time; and
d) optionally comparing an average improvement in visual acuity across patients in the subgroups within each subset.

54. The method of claim 53, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

55. The method of claim 53 or 54, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

56. The method of any claims 53-55, wherein significant impairment is characterized by the fact that the patient has a visual acuity below 23.5% of normal, below 29.4% of normal, below 35.3% of normal, below 47.0% of normal, below 50% of normal, below 50.5% of normal, below 52.9% of normal, below 55% of normal, below 57.6% of normal, or below 58% of normal.

57. The method of any of claims 53-56, wherein assessing visual acuity comprises using an eye chart, such as a Snellen chart, an early treatment of diabetic retinopathy study (ETDRS) chart, or a multiple letter acuity chart (MLAC).

58. The method of any of claims 53-57, wherein the macular edema is diabetic macular edema.

59. A method for assessing the efficacy of a candidate therapeutic treatment for macular edema, comprising
a) obtaining patient characteristic data for a plurality of candidate patients diagnosed with macular edema, the patient characteristic data including the length of time for which the patient has been diagnosed with macular edema and optionally the level of impairment of visual acuity;
b) selecting test patients from the candidate patients based in part on the length of time for which each patient has been diagnosed with macular edema and, optionally, based in part on the significance of impairment of visual acuity;
c) dividing the test patients into a first plurality of control patients and a second plurality of treatment patients;
d) assessing the visual acuity of each patient;
e) administering the candidate therapeutic treatment to the treatment patients but not to the control patients;
f) assessing the visual acuity of each of the patients at a predetermined time point after administering the candidate therapeutic treatment to the treatment patients;
g) comparing the visual acuities of each patient as measured before and after administering the candidate therapeutic treatment to the treatment patients to assess improvement in visual acuity between the assessments; and
h) comparing an average improvement in visual acuity across treatment patients with an average improvement in visual acuity among control patients, thereby assessing the efficacy of the candidate therapeutic treatment.

60. The method of claim 59, wherein selecting test patients comprises selecting test patients such that at least 50% of the test patients have been diagnosed with macular edema for at least a predetermined period of time or, optionally, suffer from significant impairment of visual acuity.

61. The method of claim 59 or 60, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

62. The method of any claims 59-61, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

63. The method of any claims 59-62, wherein significant impairment is characterized by the fact that the patient has a visual acuity below 23.5% of normal, below 29.4% of normal, below 35.3% of normal, below 47.0% of normal, below 50% of normal, below 50.5% of normal, below 52.9% of normal, below 55% of normal, below 57.6% of normal, or below 58% of normal.

64. The method of any of claims 59-63, wherein assessing visual acuity comprises using an eye chart, such as a Snellen chart, an early treatment of diabetic retinopathy study (ETDRS) chart, or a multiple letter acuity chart (MLAC).

65. The method of any of claims 59-64, wherein the macular edema is diabetic macular edema.

66. A method for comparing the efficacy of a therapeutic treatment in different subsets of macular edema patients, comprising
a) assessing the visual acuity of each of a first plurality of patients and a second plurality of patients, wherein the patients in the first plurality have experienced symptoms of macular edema for at least a predetermined period of time and the patients in the second plurality have experienced symptoms of macular edema for less than the predetermined period of time, optionally wherein each plurality is subdivided into two subgroups, wherein one subgroup consists of patients diagnosed as having significant impairment of visual acuity and the second subgroup consists of patients diagnosed as not having significant impairment of visual acuity;
b) administering a therapeutic treatment to the first and second pluralities of patients;
c) assessing the visual acuity of each of the patients at a predetermined time point after administration;
d) comparing the visual acuities of each patient as measured before and after the therapeutic treatment to assess improvement in visual acuity during the therapeutic treatment;
e) comparing an average improvement in visual acuity across patients in the first plurality with an average improvement in visual acuity among patients in the second plurality, thereby comparing the efficacy of the therapeutic treatment in patients having experienced symptoms of macular edema for at least a predetermined period of time to the efficacy of the therapeutic treatment in patients having experienced symptoms of macular edema for less than the predetermined period of time; and
f) optionally comparing an average improvement in visual acuity across patients in the subgroups within each plurality.

67. The method of claim 66, wherein the predetermined time point is at least one year after administering the therapeutic treatment.

68. The method of claim 66 or 67, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

69. The method of any claims 66-68, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

70. The method of any claims 66-69, wherein significant impairment is characterized by the fact that the patient has a visual acuity below 23.5% of normal, below 29.4% of normal, below 35.3% of normal, below 47.0% of normal, below 50% of normal, below 50.5% of normal, below 52.9% of normal, below 55% of normal, below 57.6% of normal, or below 58% of normal.

71. The method of any of claims 66-70, wherein assessing visual acuity comprises using an eye chart, such as a Snellen chart, an early treatment of diabetic retinopathy study (ETDRS) chart, or a multiple letter acuity chart (MLAC).

72. The method of any of claims 66-71, wherein the macular edema is diabetic macular edema.

73. A method for comparing the efficacy of a therapeutic treatment in different subsets of macular edema patients, comprising
a) providing data representative of improvement in visual acuity before and after administration of a therapeutic treatment for a plurality of macular edema patients;
b) dividing the data into a first subset representative of patients having experienced symptoms of macular edema for at least a predetermined period of time and a second subset representative of patients having experienced symptoms of macular edema for less than the predetermined period of time, optionally wherein each subset is subdivided into two subgroups, wherein one subgroup consists of patients diagnosed as having significant impairment of visual acuity and the second subgroup consists of patients diagnosed as not having significant impairment of visual acuity;
c) comparing an average improvement in visual acuity across patients in the first subset with an average improvement in visual acuity among patients in the second subset, thereby comparing the efficacy of the therapeutic treatment in patients having experienced symptoms of macular edema for at least a predetermined period of time to the efficacy of the therapeutic treatment in patients having experienced symptoms of macular edema for less than the predetermined period of time; and
d) optionally comparing an average improvement in visual acuity across patients in the subgroups within each subset.

74. The method of claim 73, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

75. The method of claim 73 or 74, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

76. The method of any claims 73-75, wherein significant impairment is characterized by the fact that the patient has a visual acuity below 23.5% of normal, below 29.4% of normal, below 35.3% of normal, below 47.0% of normal, below 50% of normal, below 50.5% of normal, below 52.9% of normal, below 55% of normal, below 57.6% of normal, or below 58% of normal.

77. The method of any of claims 73-76, wherein assessing visual acuity comprises using an eye chart, such as a Snellen chart, an early treatment of diabetic retinopathy study (ETDRS) chart, or a multiple letter acuity chart (MLAC).

78. The method of any of claims 73-77, wherein the macular edema is diabetic macular edema.

79. A method for assessing the efficacy of a candidate therapeutic treatment for macular edema, comprising
a) obtaining patient characteristic data for a plurality of candidate patients having experienced symptoms of macular edema, the patient characteristic data including the length of time for which the patient has experienced symptoms of macular edema and optionally the patient's visual acuity;
b) selecting test patients from the candidate patients based in part on the length of time for which each patient has experienced symptoms of macular edema and, optionally, based in part on the significance of impairment of visual acuity;
c) dividing the test patients into a first plurality of control patients and a second plurality of treatment patients;
d) assessing the visual acuity of each patient;
e) administering the candidate therapeutic treatment to the treatment patients but not to the control patients;
f) assessing the visual acuity of each of the patients at a predetermined time point after administering the candidate therapeutic treatment to the treatment patients;
g) comparing the visual acuities of each patient as measured before and after administering the candidate therapeutic treatment to the treatment patients to assess improvement in visual acuity between the assessments; and
h) comparing an average improvement in visual acuity across treatment patients with an average improvement in visual acuity among control patients, thereby assessing the efficacy of the candidate therapeutic treatment.

80. The method of claim 79, wherein selecting test patients comprises selecting test patients such that at least 50% of the test patients have experienced symptoms of macular edema for at least a predetermined period of time or, optionally, suffer from significant impairment of visual acuity.

81. The method of claim 79 or 80, wherein the predetermined period of time is approximately: 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 27 months, 30 months, 33 months, 36 months, 39 months, 42 months, 45 months, or 48 months.

82. The method of any claims 79-81, wherein significant impairment is characterized by the fact that the patient, on an early treatment of diabetic retinopathy study (ETDRS) chart, can read no more than no more than 19 letters, no more than 20 letters, no more than 21 letters, no more than 22 letters, no more than 23 letters, no more than 24 letters, no more than 25 letters, no more than 26 letters, no more than 27 letters, no more than 28 letters, no more than 29 letters, no more than 30 letters, no more than 31 letters, no more than 32 letters, no more than 33 letters, no more than 34 letters, no more than 35 letters, no more than 36 letters, no more than 37 letters, no more than 38 letters, no more than 39 letters, no more than 40 letters, no more than 41 letters, no more than 42 letters, no more than 43 letters, no more than 44 letters, no more than 45 letters, no more than 46 letters, no more than 47 letters, no more than 48 letters or no more than 49 letters.

83. The method of any claims 79-82, wherein significant impairment is characterized by the fact that the patient has a visual acuity below 23.5% of normal, below 29.4% of normal, below 35.3% of normal, below 47.0% of normal, below 50% of normal, below 50.5% of normal, below 52.9% of normal, below 55% of normal, below 57.6% of normal, or below 58% of normal.

84. The method of any of claims 79-83, wherein assessing visual acuity comprises using an eye chart, such as a Snellen chart, an early treatment of diabetic retinopathy study (ETDRS) chart, or a multiple letter acuity chart (MLAC).

85. The method of any of claims 79-84, wherein the macular edema is diabetic macular edema.
